(19) United States
(12) Patent Application Publication
(10) Pub. No.: US 2012/0129772 A1
(43) Pub. Date: May 24, 2012

(54) SOLUBLE IGF RECEPTORS AS ANTI-ANGIOGENIC AGENTS

(75) Inventors: Pnina Brodt, Montreal (CA); Zhipeng You, Montreal (CA)

(73) Assignee: THE ROYAL INSTITUTION FOR THE ADVANCEMENT OF LEARNING/McGILL UNIVERSITY, Montreal, QC (unknown)

(21) Appl. No.: 13/056,360

(22) PCT Filed: Jul. 28, 2009
(86) PCT No.: PCT/CA09/01060

§ 371 Date: Jan. 28, 2011

Related U.S. Application Data
(60) Provisional application No. 61/084,393, filed on Jul. 29, 2008.
(60) Provisional application No. 61/180,150, filed on May 21, 2009.

(51) Int. Cl. A61K 38/30; A61P 35/00; A61P 35/04; A61P 9/00

(52) U.S. Cl. 514/8.5

(57) ABSTRACT

There is disclosed a method of inhibiting angiogenesis in a patient having an angiogenic associated disorder comprising administering to said patient a therapeutically effective amount of a soluble IGF-IR protein. The use of such soluble IGF-IR protein for inhibiting angiogenesis in a patient is also disclosed.

### TECHNICAL FIELD

The present invention relates to soluble IGF receptor as anti-angiogenic agents.

### BACKGROUND OF THE INVENTION

The ability of cancer cells to detach from the primary tumor and establish metastases in secondary organ sites remains the greatest challenge to the management of malignant disease. The liver is a major site of metastasis for some of the most prevalent human malignancies, particularly carcinomas of the upper and lower gastrointestinal (GI) tract. At present, surgical resection is the only curative option for liver metastases but its success rate is partial, producing a 25-30%, 5 year disease-free survival rate for malignancies such as colorectal carcinoma (Wei, et al., 2006, Ann Surg Oncol, 13: 668-676). There is therefore a need for new therapeutic strategies that will prevent, and improve cure rates for hepatic metastases.

The receptor for the type I insulin like growth factor (IGF-IR) plays a critical role in malignant progression. Increased expression of IGF-IR and/or its ligands has been documented in many human malignancies and high plasma IGF-I levels were identified as a potential risk factor for malignancies such as breast, prostate and colon carcinomas (Samani et al., 2007, Endocr Rev, 28: 20-47). Recent data have shown that the IGF axis promotes tumor invasion and metastasis through several mechanisms, and it has been identified as a determinant of metastasis to several organ sites, particularly the lymph nodes and the liver (Long et al., 1998, Exp Cell Res, 238: 116-121; Wei, et al., 2006, Ann Surg Oncol, 13: 668-676; Samani et al., 2007, Endocr Rev, 28: 20-47; Reinmuth et al., 2002, Clin Cancer Res, 8: 3259-3269). The IGF receptor can affect metastasis by regulating tumor cell survival and proliferation in secondary sites and also by promoting angiogenesis and lymphangiogenesis either through direct action on the endothelial cells or by transcriptional regulation of vascular endothelial growth factors (VEGF) A and C (LeRoith et al., 1992, NIH conference. Insulin-like growth factors in health and disease. Ann Intern Med 116: 854-862). Pre-clinical animal studies identified the IGF-IR as a target for anti-cancer therapy in various tumor types and several IGF-IR inhibitors have recently entered phase I and II clinical trials for the treatment of disseminated cancer.

The IGF-IR ligands include three structurally homologous peptides IGF-I, IGF-II and insulin, but the receptor binds IGF-I with the highest affinity. The major site of endocrine production for IGF-I and IGF-II is the liver (Werner & Le Roith, 2000, Cell Mol Life Sci 57: 932-942), but autocrine/paracrine IGF-I production has been documented in extra-hepatic sites such as heart, muscle, fat, spleen and kidney. The physiological activities and bioavailability of IGF-I and IGF-II are modulated through their association with 6 secreted, high-affinity binding proteins (IGFBP1-6).

Decoy receptors for treatment of malignant disease have been taught as a potential therapeutic treatment. Vehicles for such decoy should ideally be developed in order to deliver therapeutically effective concentrations of the decoy receptor in a sustained manner into the tumor site.

It would be highly desirable to be provided with new therapeutic strategies that will prevent, and improve cure rates for metastases such as hepatic metastases. Furthermore, it would be highly desirable to be provided with decoy receptors for treatment of malignant disease.

### SUMMARY OF THE INVENTION

In accordance with the present invention there is now provided a method of inhibiting angiogenesis in a subject having an angiogenic associated disorder comprising administering to the subject a therapeutically effective amount of a soluble IGF-IR protein comprising the extracellular domain of IGF-IR having the amino acid sequence of SEQ ID NO: 3 or a biologically active fragment thereof.

In an alternate embodiment, there is also provided a method of inhibiting angiogenesis in a subject having an angiogenic associated disorder comprising administering to the subject a stromal cell genetically modified to express a soluble IGF-IR protein comprising the extracellular domain of IGF-IR having the amino acid sequence of SEQ ID NO: 3 or a biologically active fragment thereof.

In a further embodiment, there is provided the use of a soluble IGF-IR protein comprising the extracellular domain of IGF-IR having the amino acid sequence of SEQ ID NO: 3 or a biologically active fragment thereof for inhibiting angiogenesis in a subject having an angiogenic associated disorder.

There is also provided the use of a stromal cell genetically modified to express a soluble IGF-IR protein comprising the extracellular domain of IGF-IR having the amino acid sequence of SEQ ID NO: 3 or a biologically active fragment thereof for inhibiting angiogenesis in a subject having an angiogenic associated disorder.

Alternatively, there is disclosed a method of preventing or treating an angiogenic associated disorder in a subject, the method comprising administering a soluble IGF-IR protein comprising the extracellular domain of IGF-IR having the amino acid sequence of SEQ ID NO: 3 or a biologically active fragment thereof, wherein angiogenesis is inhibited in the subject, such that the angiogenic associated disorder is prevented or treated.

In a further embodiment, there is provided a method of preventing or treating tumor metastasis, colorectal carcinoma, lung carcinoma or hepatic cancer in a subject, the method comprising administering a soluble IGF-IR protein comprising the extracellular domain of IGF-IR having the amino acid sequence of SEQ ID NO: 3 or a biologically active fragment thereof, wherein angiogenesis is inhibited in the subject, such that the tumor metastasis, colorectal carcinoma, lung carcinoma or hepatic cancer is prevented or treated.

There is also provided a pharmaceutical composition for inhibiting angiogenesis in a subject, comprising a soluble IGF-IR protein comprising the extracellular domain of IGF-IR having the amino acid sequence of SEQ ID NO: 3 or a biologically active fragment thereof; and a pharmaceutically acceptable carrier.

In a further embodiment, there is provided the use of a soluble IGF-IR protein comprising the extracellular domain of IGF-IR having the amino acid sequence of SEQ ID NO: 3 or a biologically active fragment thereof in the manufacture of a medicament for inhibiting angiogenesis in a subject having an angiogenic associated disorder.

Preferably, the soluble IGF-IR protein forms the tetrameric structure of SEQ ID NO: 3.

Furthermore, the soluble IGF-IR protein comprises or consists of SEQ ID NO: 1 or a biologically active fragment or analog thereof.

In a preferred embodiment, the angiogenic associated disorder is cancer, such as tumor metastasis, colorectal carcinoma, lung carcinoma, hepatic cancer. Preferably, hepatic cancer is liver metastasis.

In another embodiment, the method or use described herein encompass the soluble IGF-IR protein being administered in combination with another angiogenesis inhibitor.

In another embodiment, the method or use described herein encompass the soluble IGF-IR protein being administered via injection, such as intravenous or intraperitoneal injection.

In a further embodiment, the stromal cell is a bone marrow derived mesenchymal stromal cell.

In another embodiment, the soluble IGF-IR protein retains the disulfide bonds of SEQ ID NO: 3 and/or high affinity ligand binding.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates a 6% SDS-polyacrylamide gel under reducing (A) or non-reducing (B) conditions using 20 mg protein per lane, wherein proteins were detected with a rabbit antibody to the human IGF-IR followed by a peroxidase-conjugated donkey anti-rabbit IgG. Lanes 1 corresponds to BMSC^{GFP}, lanes 2 corresponds to BMSC^{EPO} and lanes 3 correspond to BMSC^{sIGFIR}. Numbers on the left denote the positions of MW markers.

FIG. 2 illustrates the detection of circulating soluble IGF-IR in mice implanted with genetically engineered marrow stromal cells, wherein ten million BMSC cells were embedded in Matrigel and implanted subcutaneously into syngeneic C57Bl/6 (A) or athymic (B) mice. Each value represents the mean (and SD) of 3-33 individual measurements performed on the indicated day post MSC implantation. In (A) and (B), the curve with points represented with (♦) were obtained with mice implemented with BMSC^{sIGFIR}; points represented with (▴) were obtained with mice implemented with BMSC^{EPO}; and points represented with (▪) were obtained with mice implemented with BMSC^{GFP}.

FIG. 3 illustrates the plasma concentration of circulating sIGFIR⁹³³/IGF-I in (A) complexes semi-quantified by a combined ELISA using the mouse anti-human IGF-IR antibody to capture the complexes, a biotinylated goat anti-mouse IGF-I antibody for detection and a recombinant human IGFIR standard curve for quantification. Shown are the means (and SD) of values obtained from 3 different plasma pools each derived from at least 6 mice. In (B), it is shown the plasma levels of IGF-I (♦) measured using an RIA. Known standards were place in each analysis and pooled mouse sera from normal 16 wk old C57Bl/6 mice was used as an additional control (▪). Shown are the means and SD of 7-15 individual samples analyzed per each time point.

FIG. 4 illustrates syngeneic female C57Bl/6 (A-D) or nude (E) mice were implanted with 10⁷ genetically engineered or control BMSC embedded in Matrigel. Nine (A,B) or 14 (C-E) days later the mice were inoculated via the intrasplenic/portal route with 10⁵ H-59 (A-C), 5×10⁴ MC-38 (D) or 2×10⁵ KM12SM (E) cells. Shown in (A) and (B) are the pooled data of 2 experiments each performed using a saline (lanes 1), BMSC^{GFP} (lanes 2) and BMSC^{sIGFIR} (lanes 3). Shown in C are the pooled results of 3 experiments using a saline (lane 1), BMSC^{GFP} (lane 2) and BMSC^{sIGFIR} (lane 3). In (D) and (E), it is shown individual experiments using the indicated numbers of mice per group, using saline (lanes 1), BMSC^{GFP} (lanes 2) and BMSC^{sIGFIR} (lanes 3). Shown in (F) are representative livers from one experiment performed with H-59 cells and depicted in panel (B), wherein line 1 represents non-treated livers, lane 2 represent BMSC^{IGFIR933} treated livers and lane 3 represents control BMSC treated livers. Shown in (G) and (H) are representative H&E stained sections obtained from formalin fixed and paraffin embedded livers of KS12SM-injected nude mice, wherein panels 1 relate to livers embedded with BMSC^{GFP} and panels 2 relate to embedded livers with BMSC^{IGFIR933}. In I, it is shown a photographic representation of wherein detectable GFP signal was detected by day 18, when all the mice were euthanized (panel 1 relating to livers embedded with BMSC^{GFP} and panel 2 relating to embedded livers with BMSC^{IGFIR933}).

FIG. 5 illustrates in (A) the amount of CD31+ microvessels per μm² in mice implemented with BMSC^{GFP} (lane 1) or BMSC^{IGFIR933} (lane 2). Shown are means and SE based on 30 individual images analyzed, p<0.0001. Shown in (B) are representative images acquired using the 40× objective. Results of a TUNEL assay performed on sections derived from the same livers are shown in (C). Shown are means and SE of the proportion of TUNEL⁺ nuclei/total nuclei seen in 12 individual images (p=0.0040). Representative images showing GFP+ tumor cells (green), total nuclei (blue), and apoptotic cell (red) and the merged images for each group are shown in (D).

FIG. 6 illustrates in (A) polyacrylamide gel electrophoresis (PAGE) and in (B) Western blotting to confirm the purity of sIGFIR purified using FPLC and an Ni-NTA column.

FIG. 7 shows plasma sIGFIR levels in injected mice, wherein plasma sIGFIR concentrations were measured by ELISA.

FIG. 8 shows liver metastases in mice injected with sIGFIR, wherein liver metastases were enumerated 14 days following the instrasplenic/portal inoculation of tumor H-59 cells, wherein the number of metastases per liver is shown in (A), representative livers are shown in (B), and n is the number of animals injected per treatment group.

FIG. 9 shows that incubation of tumor cells in vitro with sIGFIR increases anoikis of the tumor cells.

It will be noted that throughout the appended drawings, like features are identified by like reference numerals.

### DETAILED DESCRIPTION

The present invention provides the use of soluble IGF receptors as anti-angiogenic agents.

As used herein, the term “angiogenesis” means the proliferation of new blood vessels that penetrate into tissues or organs or into cancerous growths. Under normal physiological conditions, humans or animals undergo angiogenesis only in very restricted situations. For example, angiogenesis is normally observed in wound healing, fetal and embryonic development and formation of the corpus luteum, endometrium and placenta.

Pathological angiogenesis occurs in a number of disease states, for example, tumor metastasis and abnormal growth by endothelial cells, and supports the pathological damages seen in these conditions. The diverse pathological disease states in which abnormal angiogenesis is present have been grouped together as “angiogenic dependent” or “angiogenic associated” disorders.

Angiogenesis is tightly regulated by both positive and negative signals. Angiogenic stimulators, such as fibroblast growth factor (FGF) and vascular endothelial growth factor (VEGF), are potent mitogens for endothelial cell proliferation and strong chemoattractants for endothelial cell migration. These positive regulators can promote neovascularization to sustain the expansion of both primary and metastatic tumors. Among the negative regulators described to date, angiostatin ranks as one of the most effective endogenous inhibitors of angiogenesis.

The receptor for the type 1 insulin-like growth factor (IGF-IR) has been identified as a target for anti-cancer therapy.

The IGF-IR is a heterotetrameric receptor tyrosine kinase (RTK) consisting of two 130-135 kDa α and two 90-95 kDa β chains, with several α-α and α-β disulfide bridges. It is synthesized as a polypeptide chain of 1367 amino acids that is glycosylated and proteolytically cleaved into α- and β-subunits that dimerize to form a tetramer. The ligand binding domain is on the extracellular α subunit, while the β subunit consists of an extracellular portion linked to the α subunit through disulfide bonds, a transmembrane domain and a cytoplasmic portion with a kinase domain and several critical tyrosines and serine involved in transmission of ligand-induced signals (Samani et al., 2004, Cancer Research, 64: 3380-3385).

IGF-IR expression and function are critical for liver metastases formation in different tumor types. Tumor cells engineered to express a soluble form of IGF-IR (sIGFIR) lost the ability to metastasize to the liver (Samani et al., 2004, Cancer Res, 64: 3380-3385).

An effective strategy for blocking the action of cellular receptor tyrosine kinases (RTKs) is the use of soluble variants of these receptors that can bind and reduce ligand bioavailability to the cognate receptor in a highly specific manner (Kong & Crystal, 1998, J Natl Cancer Inst, 90: 273-286; Tseng et al., 2002, Surgery, 132: 857-865; Trieu et al., 2004, Cancer Res, 64: 3271-3275). One example for successful application of this strategy is the production of the VEGFR1/VEGFR2-Fc decoy receptor (the VEGF Trap) that is currently in clinical trials as a new type of anti-angiogenic, anti-cancer drug (Rudge et al., 2005, Cold Spring Harb Symp Quant Biol, 70: 411-418).

U.S. Pat. No. 6,084,085 discloses the use of soluble IGF-IR proteins for inducing apoptosis and inhibiting tumorigenesis. The soluble IGF-IR proteins disclosed in U.S. Pat. No. 6,084,085 comprise up to about 800 amino acids of the N-terminus of IGF-IR, such that the C-terminus transmembrane domain is completely deleted or is present to the extent that the protein comprising a portion of the transmembrane domain is not able to be anchored in the cell membrane. U.S. Pat. No. 6,084,085 disclosed the preferred use of a protein comprising the N-terminal 486 amino acids of IGF-IR without a signal peptide (amino acids 1 to 486), or comprising 516 amino acids with a signal peptide (amino acids −30 to 486). The proteins disclosed in U.S. Pat. No. 6,084,085 do not include the regions of the IGF-IR required for dimerization and multimerization.

We report herein for the first time that a soluble IGF-IR receptor has anti-angiogenic properties. There is provided herein a therapeutic approach for the prevention and/or treatment of angiogenic dependent or angiogenic associated disorders, e.g. hepatic metastases, based on the sustained in vivo delivery of soluble receptor acting as an anti-angiogenic agent. To this end, autologous bone marrow-derived mesenchymal stromal cells have been genetically engineered to produce high levels of the soluble receptor and these were embedded in Matrigel™ and implanted subcutaneously into mice prior to the intrasplenic/portal inoculation of highly metastatic tumor cells. The soluble receptor has also been purified and injected into mice, e.g. intravenously or intraperitoneally, prior to the intrasplenic portal inoculation of highly metastatic tumor cells.

Soluble IGF-IR receptor is referred to herein as sIGFIR, sIGF-IR, soluble IGFIR and soluble IGF-IR, and these terms are used interchangeably.

The term “genetically-engineered stromal cell” or “transgenic stromal cells” as used herein is intended to mean a stromal cell into which an exogenous gene has been introduced by retroviral infection or other means well known to those of ordinary skill in the art. The term “genetically-engineered” may also be intended to mean transfected, transformed, transgenic, infected, or transduced.

The term “ex vivo gene therapy” is intended to mean the in vitro transfection or retroviral infection of stromal cells to form transfected stromal cells prior to implantation into a mammal.

The expression “transduction of bone marrow stromal cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced bone marrow stromal cell. A bone marrow stromal cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

The term “stromal cells” as used herein is intended to mean marrow-derived fibroblast-like cells defined by their ability to adhere and proliferate in tissue-culture treated petri dishes with or without other cells and/or elements found in loose connective tissue, including but not limited to, endothelial cells, pericytes, macrophages, monocytes, plasma cells, mast cells, adipocytes, etc.

Liver-metastasizing lung carcinoma cells genetically engineered to produce a 933-amino-acid residue (identified as sIGFIR933; SEQ ID NO: 1) soluble peptide spanning the entire extracellular domain of the full-length IGF-IR (SEQ ID NO: 3) lost all IGF-IR regulated functions and failed to produce liver metastases in a high proportion of mice inoculated via the intrasplenic/portal route, resulting in markedly increased long term, disease free survival. Immunohistochemical analysis performed on livers derived from the injected animals, revealed wide-spread apoptosis in tumor cells expressing sIGFIR⁹³³.

It is also provided herein that mice implanted with autologous bone marrow stromal cells engineered to produce sIGFIR⁹³³ had measurable circulating levels of the protein and this resulted in dramatically reduced numbers of hepatic metastases of three different, highly metastatic tumors. Thus, reduction in metastases was a consequence of a marked decrease in tumor-induced angiogenesis and increased tumor apoptosis during the early stages of liver colonization.

One promising therapeutic strategy disclosed herein and that is becoming a clinical reality is the use of autologous cells that have a regenerative capacity and can be genetically engineered to produce effective concentrations of the desired protein (Buckley, 2000, Nat Med, 6: 623-624; Cavazzana-Calvo et al., 2000, Science, 288: 669-672; Dobson, 2000, Bmj, 320: 1225; Stephenson, 2000, Jama, 283: 589-590). Bone marrow derived mesenchymal stromal cells (BMSC) have been used to this end and have several advantages as delivery vehicles: they are abundant and available in humans of all age groups, can be harvested with minimal morbidity and discomfort, have a proliferative capacity, can be genetically engineered with reasonable efficiency and are easy to re-implant in the donor without “toxic” conditioning regimen such as radiotherapy, chemotherapy or immunosuppression. BMSCs have been validated as an efficient autologous cellular vehicle for the secretion of various beneficial proteins in vivo in both immunodeficient and immunocompetent hosts and could become an effective tool for protein delivery in clinical practice (Stagg & Galipeau, 2007, Handb Exp Pharmacol, 45-66). Consequently, there is disclosed herein the use of BMSCs autologous cells as vehicles for the secretion of sIGFIR⁹³³. Any other vehicle for expressing protein known in the art is also encompassed herein, and thus BMSCs represent one embodiment of the present invention, which is not restricted to BMSCs.

It is disclosed herein that the genetically altered stromal cells produced and secreted high levels of the soluble receptor that are detectable in the serum for up to several weeks post implantation. In mice implanted with these cells, but not with control stromal cells, marked reductions in the number of hepatic metastases are seen following the injection of murine colorectal carcinoma MC-38 (up to 82% reduced) and lung carcinoma H-59 (up to 95%) cells, as well as human colorectal carcinoma KM12SM cells (up to 64%) that were inoculated into athymic nude mice.

Analysis of liver cryostat sections by immunohistochemistry and confocal microscopy revealed in mice implanted with sIGFIR-producing stromal cells a significant reduction in intra-lesional angiogenesis as reflected in reduced microvessel density and this coincided with a 16 fold increase in the number of tumor cells undergoing apoptosis, demonstrating that the soluble receptor acted as a decoy to abort IGF-IR functions during the early stages of metastatic growth. These results identify sIGFIR as a potent anti-angiogenic agent and also as a therapeutic, anti-metastatic agent.

It is also disclosed herein that purified sIGFIR protein injected into mice reduced liver metastases from subsequently injected tumor H-59 cells, further confirming the use of sIGFIR as a therapeutic, anti-metastatic agent. In addition, incubation of tumor cells in vitro with sIGFIR protein increased apoptosis of the tumor cells.

Also encompassed within the scope of the present invention are sIGFIR933 variations and fragments, including biologically active fragments, and biologically active analogs involving amino acid deletions, additions and/or substitutions. “Biologically active fragment” includes fragments of sIGFIR933 that maintain essentially the same biological activity of the sIGFIR933 from which the fragment is derived. “Biologically active analogs” includes variations of sIGFIR933 region(s) that do not materially alter the biological activity (i.e., anti-angiogenic activity) of the sIGFIR933 from which the analog is derived. Included within the scope of the invention are changes made to the sIGFIR933 and sIGFIR933 fragment(s) that increase anti-angiogenic activity.

In one embodiment, the invention also encompasses a biologically active fragment of SEQ ID NO: 3 which retains the ability to form α-α and α-β disulfide bridges. Particularly, a biologically active fragment of SEQ ID NO: 3 may comprise α- and β-subunits that dimerize to form a tetramer. In another embodiment, the invention encompasses a soluble IGF-IR protein comprising a biologically active fragment of SEQ ID NO: 3 which retains the disulfide bonds in the extracellular domain of the native (wild-type) receptor and/or mimics the 3D conformation of the native (wild-type) receptor. In another embodiment, a biologically active fragment retains high affinity ligand binding.

Preferred analogs include those that incorporate modifications to the sIGFIR⁹³³ region(s) and/or fragment(s). The resulting sequences differ from the wild-type sequence by one or more conservative amino acid substitutions or by one or more non-conservative amino acid substitutions, deletions or insertions, wherein the substitutions, deletions or insertions do not abolish the biological activity of the wild-type sequence. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

Other analogs within the invention are those with modifications which increase protein or peptide stability; such analogs may contain, for example, one or more non-peptide bonds (which replace the peptide bonds) in the protein or peptide sequence. Also included are analogs that include residues other than naturally occurring L-amino acids, e.g., D-amino acids or non-naturally occurring or synthetic amino acids, e.g., β or γ amino acids. Further within the invention is the addition of peptide sequences such as but not restricted to the Fc portion of the immunoglobulin G protein.

Also disclosed is a composition comprising the sIGFIR⁹³³ described herein (or a biologically active fragment or analog thereof), which is useful to treat angiogenic-dependent or angiogenic-associated disorders. The present invention includes the method of treating an angiogenic-dependent or angiogenic-associated disorder with an effective amount of a composition comprising a sIGFIR⁹³³. Such compositions may also include a pharmaceutically acceptable carrier, adjuvant or vehicle. In another aspect, the compositions and methods of the invention are used to inhibit angiogenesis in a subject in need thereof, e.g. in a subject having an angiogenic dependent or angiogenic associated disorder. In one aspect, the angiogenic associated disorder is tumor metastasis, colorectal carcinoma, lung carcinoma or hepatic cancer or hepatic metastases.

Angiogenic dependent and/or angiogenic associated disorders includes, but are not limited to, solid tumors, blood born tumors such as leukemias; tumor metastasis; benign tumors, for example, hemangiomas, acoustic acuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation. The compositions of the present invention are useful in treatment of disease of excessive or abnormal stimulation of endothelial cells. These disorders include, but are not limited to, intestinal adhesions, atherosclerosis, scleroderma, and hypertrophic scars, i.e., keloids. The compositions can also be used as birth control agents by preventing vascularization required for embryo implantation.

The compositions and methods of the present invention may be used in combination with other compositions, methods and/or procedures for the treatment of angiogenic-dependent or angiogenic-associated disorders. For example, a tumor may be treated conventionally with surgery, radiation or chemotherapy, and then compositions comprising a sIGFIR⁹³³ as disclosed herein may be subsequently administered to the patient to extend the dormancy of micrometastases and to stabilize any residual primary tumor.

The present invention also provides pharmaceutical (i.e., therapeutic) compositions comprising a sIGFIR⁹³³ (or a biologically active fragment or analog thereof), optionally in combination with at least one additional active compound, and/or any pharmaceutically acceptable carrier, adjuvant or vehicle. “Additional active compounds” encompasses, but is not limited to, an agent or agents such as an immunosuppressant or anti-cancer agent.

The term “pharmaceutically acceptable carrier, adjuvant or vehicle” refers to a carrier, adjuvant or vehicle that may be administered to a subject, incorporated into a composition of the present invention, and which does not destroy the pharmacological activity thereof. Pharmaceutically acceptable carriers, adjuvants and vehicles that may be used in the pharmaceutical compositions of the present invention include, but are not limited to, the following: ion exchangers, alumina, aluminum stearate, lecithin, self-emulsifying drug delivery systems (“SEDDS”), surfactants used in pharmaceutical dosage forms such as Tweens or other similar polymeric delivery matrices, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β- and γ-cyclodextrin, or chemically modified derivatives such as hydroxyalkylcyclodextrins, including 2- and 3-hydroxypropyl-β-cyclodextrins, or other solubilized derivatives may also be used to enhance delivery of the compositions of the present invention.

The compositions of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The compositions of the present invention may be administered by any suitable means, for example, orally, such as in the form of tablets, capsules, granules or powders; sublingually; buccally; parenterally, such as by subcutaneous, intravenous, intramuscular, intraperitoneal or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compositions may, for example, be administered in a form suitable for immediate release or extended release. Immediate release or extended release may be achieved by the use of suitable pharmaceutical compositions, or, particularly in the case of extended release, by the use of devices such as subcutaneous implants or osmotic pumps.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The present compounds may also be delivered through the oral cavity by sublingual and/or buccal administration. Molded tablets, compressed tablets or freeze-dried tablets are exemplary forms which may be used. Exemplary compositions include those formulating the present compositions with fast dissolving diluents such as mannitol, lactose, sucrose and/or cyclodextrins. Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

The effective amount of a compound of the present invention may be determined by one of ordinary skill in the art, and includes exemplary dosage amounts for an adult human of from about 0.1 to 500 mg/kg of body weight of active compound per day, which may be administered in a single dose or in the form of individual divided doses, such as from 1 to 5 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion and clearance, drug combination, and severity of the particular condition. Preferred subjects for treatment include animals, most preferably mammalian species such as humans, and domestic animals such as dogs, cats and the like, subject to angiogenic dependent or angiogenic associated disorders.

The compositions of the present invention may be employed alone or in combination with other suitable therapeutic agents useful in the treatment of angiogenic dependent or angiogenic associated disorders, such as angiogenesis inhibitors other than those of the present invention.

The present invention will be more readily understood by referring to the following examples which are given to illustrate the invention rather than to limit its scope.

### Example I

### Genetically Engineered Autologous Bone Marrow Cells Produce a Soluble IGF-IR Protein In Vitro

To begin to evaluate the potential applications of a soluble IGFIR decoy in a therapeutic setting, autologous mesenchymal bone marrow cells (referred to herein as BMSC or MSC (the two terms are used interchangeably)) were genetically engineered by transduction with retroviral particles expressing a cDNA fragment corresponding to the first 2799 nucleotides (SEQ ID NO: 2) of a human IGF-IR cDNA that encodes the sIGFIR⁹³³ peptide. This strategy was chosen with the objective of achieving a sustained in vivo production of the soluble peptide for the duration of the animal experiments.

Construction of the pLTR-GFP-IGIR⁹³³ vector expressing a cDNA fragment corresponding to the first 2799 nucleotides (SEQ ID NO: 2) of the human IGF-IR RNA that encodes the 933-amino-acid (SEQ ID NO: 1) extracellular domain of IGF-IR downstream of the cmv promoter was as described in Samani et al. (2004, Cancer Res, 64: 3380-3385). To produce retrovirus particles expressing IGFIR⁹³³, the GP2-293 cells (ClonTech, CA, USA) were used, as per the supplier's instructions. Briefly, cell monolayers at 75% confluency were co-transfected with 5 μg of the pLTR-IGIR⁹³³ vector that also encodes the green fluorescent protein (GFP) and 5 μg of pVSV-G (ClonTech, CA, USA) using Lipofectamine™ (Invitrogen™). The cells were incubated for 48-72 hours at which time the medium was harvested, filtered and added to semi confluent BMSC cultures in 60 mm culture dishes together with 4-8 μg/ml Polybrene® (Sigma, Mo., USA). This transduction protocol was repeated several times until sIGFIR could be detected in the culture medium of BMSC^{sIGFIR} cells by Western blotting.

Subconfluent monolayers of BMSC were washed extensively to remove serum and the cells were cultured in serum-free medium for 24 hours at 37° C. The conditioned media were concentrated 30-fold and the concentrated proteins loaded on a 6% polyacrylamide gel and separated by polyacrylamide gel electrophoresis under non-reducing or reducing conditions. Immunoblotting was performed using a rabbit polyclonal antibody to human IGF-IR (Santa Cruz Biotechnology®, Santa Cruz, Calif.) diluted 1:200 and peroxides-conjugated donkey anti-rabbit IgG (Cedarlane, Hornby, Ontario, Canada) diluted 1:10,000, as secondary antibody. Protein bands were visualized using the enhanced chemiluminescence system (Roche, Basel, Switzerland).

BMSC transduced in the same manner with retroviral particles expressing the GFP cDNA only (BMSC^{GFP}) and, in some experiments, BMSC engineered to produce erythropoietin (BMSC^{EPO}) as described in Eliopoulos et al. (2000, Blood, 96: 802a) were used as controls. Both BMSC^{sIGFIR} and BMSC^{GFP} cells were sorted using a FACSCalibur™ (Beckton-Dickinson) to produce a GFP-enriched subpopulation in which >95% cells were highly fluorescent, as assessed by flow cytometry and these cells were used for all subsequent in vivo experiments.

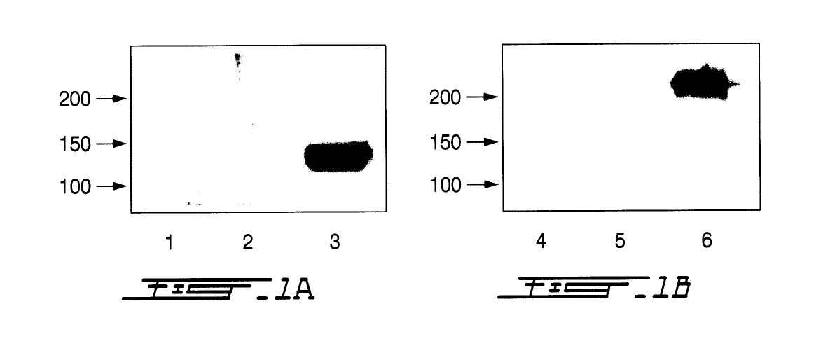

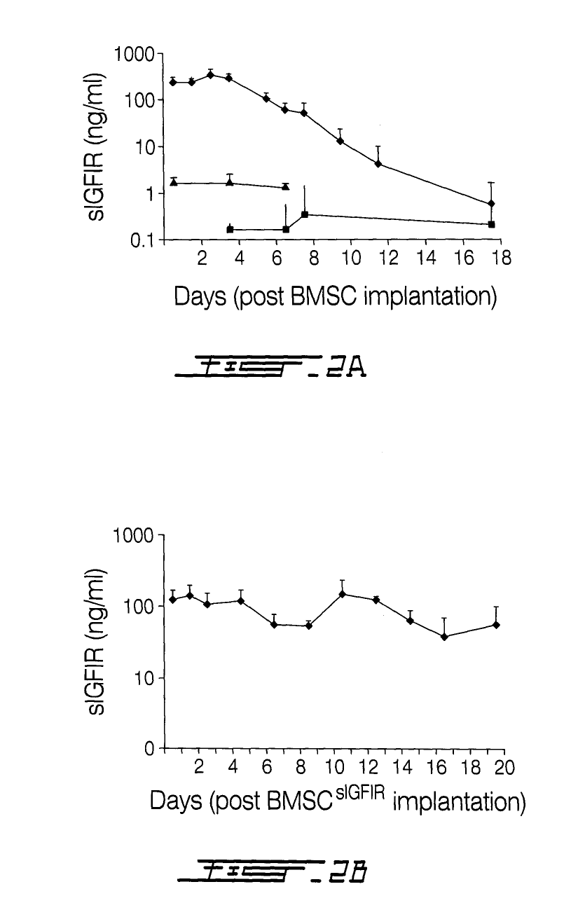

Western blotting performed with an antibody to the α subunit of the human IGF-IR (FIG. 1) revealed single bands corresponding to the α subunit (reducing conditions; panel A) or the soluble receptor tetramer (non-reducing conditions; panel B) in serum-free conditioned media harvested from these cells (BMSC^{sIGFIR}; lanes 3 and 6), but not from BMSC transduced with control retroviral particles expressing either the GFP gene alone (BMSC^{GFP}; lanes 1 and 4) or a full length erythropoietin cDNA (BMSC^{EPO}; lanes 2 and 5) (FIG. 1), confirming that the cells expressed and secreted the decoy receptor in vitro.

### Example 2

### Genetically Engineered Bone Marrow Cells Produce a Soluble IGF-IR Protein In Vivo

The ability of the disclosed cells to produce and secrete the soluble decoy in vivo was evaluated. BMSC^{sIGFIR933} and controls were embedded into Matrigel™ and implanted subcutaneously as previously described Eliopoulos et al. (2003, Gene Ther, 10: 478-489). To monitor in vivo production and measure plasma levels of the protein, blood samples were collected 3 times weekly into heparinized capillary tubes and the plasma analyzed by an ELISA for the presence of soluble hIGF-IR.

Plasma concentrations of sIGFIR⁹³³ and circulating mouse IGF-I levels were quantified using the human IGF-I R and mouse IGF-I DuoSet ELISA Development Systems (R&D system, Minneapolis, Minn.), respectively. The presence of circulating sIGFIR⁹³³/IGF-I complexes was assessed and their plasma concentration semi-quantified by a combined ELISA using the mouse anti-IGF-IR antibody (R&D system) to capture the complexes, a biotinylated goat anti mouse IGF-I antibody (R&D System) for detection and an IGF-I standard curve for quantification. In all the experiments, plasma obtained from control untreated mice was used to establish baselines. In addition, BMSC-sIGFIR⁹³³ and BMSC-GFP conditioned media were used as positive and negative controls, respectively.

All animal experiments were conducted in accordance with the guidelines of the institutional Animal Care committee. To initiate sustained in vivo production of sIGFIR⁹³³, the genetically engineered BMSC^{sIGFIR} (and BMSC^{GFP}, as controls) were dispersed with a 0.2% trypsin-EDTA solution, centrifuges and resuspended in RPMI medium. For each injection, 10⁷ cells in 50 μl RPMI were mixed with 450 μl undiluted Matrigel™ (Becton-Dickinson, Mississauga, ON, Canada) and the mixture kept at 4° C. until used. The entire volume was then implanted by subcutaneous injection into the right flank, as described in Eliopoulos et al. (2003, Gene Ther, 10: 478-489). At body temperature, the Matrigel™ implant rapidly acquired a semisolid form and it remained in the animals for the duration of the experiment. To monitor circulating levels of sIGFIR⁹³³, blood samples were collected from the saphenous vein using heparinized microhaematocrit tubes, and the plasma separated and tested by ELISA, as described above.

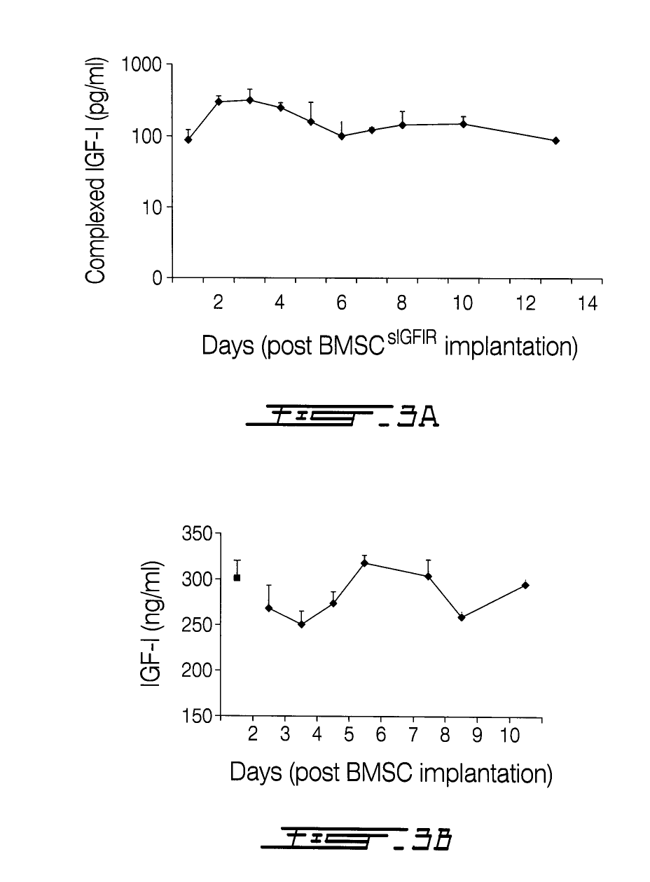

It is disclosed that within 24 hours following implantation, the sIGFIR protein could be detected in plasma obtained from mice implanted with BMSC^{sIGFIR933}. In contrast, plasma obtained from control mice implanted with either BMSC^{GFP} or BMSC^{EPO} showed the presence of the same low background levels of the peptide that could be detected in uninjected animals (FIG. 2A). The sIGFIR⁹³³ levels peaked at day 3 at approximately 300 ng/ml per mouse and were detectable for at least 18 days post-implantation at which time they measured 1-5 ng protein/ml. Hematoxylin and eosin (H&E) stained sections of paraffin embedded Matrigel™ plugs removed from the mice 22 days post implantation revealed multiple GFP+BMSC. In athymic nude mice implanted with these cells, lower plasma levels of the soluble receptor ranging from 120-150 ng/ml were initially detected at 3 days post implantation. However, protein production levels in these mice was more stable, remaining at similar high levels for at least 20 days following implantation (FIG. 2C). These results confirmed that i) the implanted BMSC were able to secrete the decoy receptor in vivo, ii) the protein accessed the circulation and iii) it was detectable for at least 3-4 weeks post implantation. The results also demonstrate that host immunity may have been involved in regulating the level and duration of sIGFIR production by the implanted bone marrow-derived stromal cells.

### Example 3

### The Soluble IGF-I Receptor Forms a Complex with Circulating IGF-I

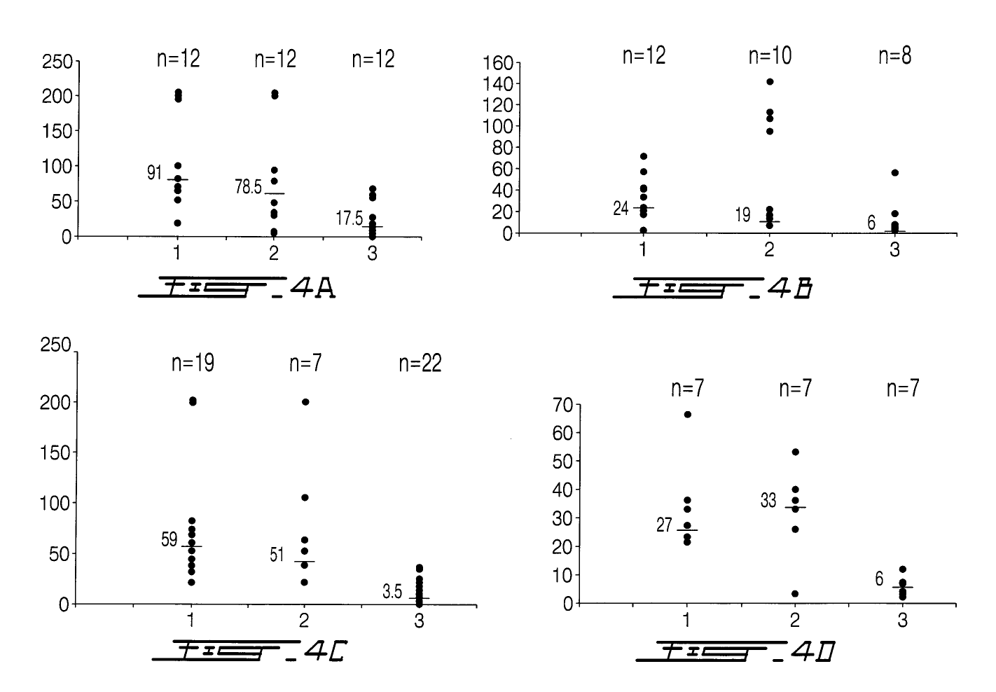

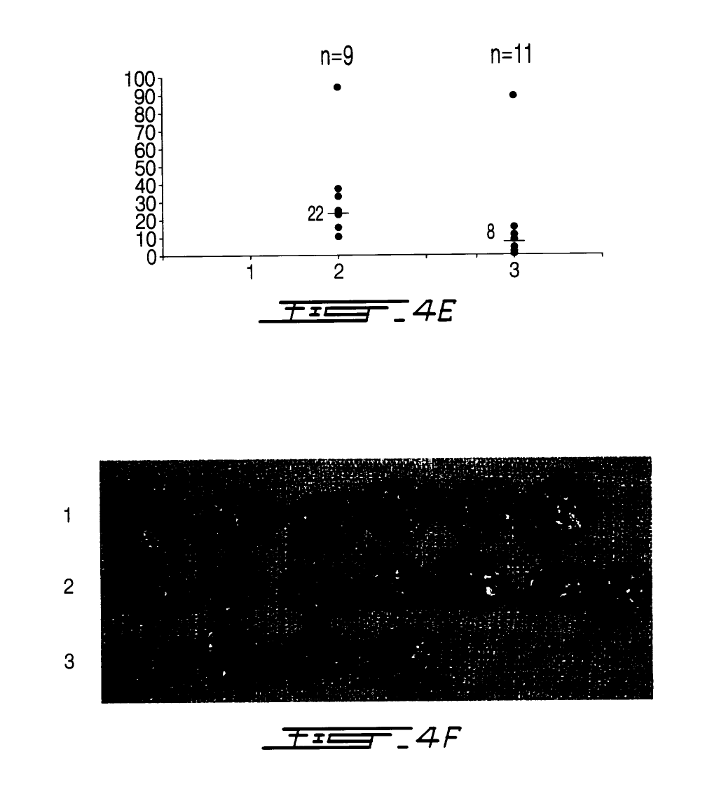

Decoy receptors can inhibit the biological activity of the cognate, membrane-bound receptors by binding and decreasing ligand bioavailability for the latter receptor (Rudge, et al., 2007, Proc Natl Acad Sci USA, 104: 18363-18370). The presence of shIGF-IR:mIGF-I complexes in the circulation of BMSC^{sIGFIR933}-implanted mice was measured using a combination ELISA test. It was found that IGF-I complexed to the soluble receptor was present in the plasma as early as 24 hr post BMSC^{sIGFIR933} implantation. The level of sIGFIR-bound IGF-I increased for the first post 3 days than declined slowly, but remained detectable for at least 2 weeks post implantation (FIG. 3A). The total circulating IGF-I levels detectable in the plasma declined initially by up to 17% relative to controls (day 3) but recovered slowly returning to control levels by day 10 post implantation (FIG. 3B). As expected, there was no evidence of complex formation in mice implanted with control BMSC.

### Example 4

### Bone Marrow Stromal Cells Producing a Soluble IGF-I Receptor Inhibit the Development of Experimental Hepatic Metastases

To analyze the effect that circulating sIGFIR and sIGFIR:IGF-1 complexes have on the ability of tumor cells to colonize the liver and establish hepatic metastases, three different tumor cell lines that are highly metastatic to the liver, i.e. the murine lung carcinoma H-59 and colorectal carcinoma MC-38 and human colorectal carcinoma KM12SM cells, were used.

Tumor H-59 is a subline of the Lewis lung carcinoma that is highly metastic to the liver (Brodt, 1986, Cancer Res, 46: 2442-2448). The cells were maintained in RPMI 1640 medium supplemented with 10% FCS and antibiotics. Murine MC-38 colon adenocarcinoma cancer cells (Yakar at al., 2006, Endocrinology, 147: 5826-5834) were maintained in Dulbecco's Modified Eagle's Medium (DMEM) supplemented with 10% fetal calf serum (Invitrogen™, GIBCO®, Ontario, Canada) and glutamine (BioSource™, Camarillo, Calif.). Human colorectal carcinoma KM12SM cells were maintained in minimal essential medium (MEM) supplemented with 10% fetal bovine serum, sodium pyruvate, nonessential amino acids, L-glutamine, vitamins (Life Technologies™, Grand Island, N.Y.), and a penicillin/streptomycin mixture (Flow Laboratories, Rockville, Md.). GP2-293 cells (ClonTech, CA, USA) and mouse bone marrow stromal cells (BMSC) were maintained in DMEM (Invitrogen™) with 10% FCS (Invitrogen™). All cells were cultured at 37° C. in a humidified incubator and a mixture of 5% CO₂ and 95% air and used within 2-4 weeks of cell recovery from frozen stocks.

Mice were first implanted with BMSC and this was followed by the injection of 5×10⁴ (MC-38), 10⁵ (H-59) or 2×10⁵ (KM12SM) tumor cells via the intrasplenic/portal route into syngeneic C57Bl/6 (H-59 and MC-38) or nude mice (KM12SM) 9-14 days later. This period was chosen based on preliminary time course experiments that revealed that the effect of the stromal cells was optimal when tumor injection was performed at least one week post BMSC implantation. The livers were removed and the macroscopic metastases enumerated prior to fixation, with the aid of a dissecting microscope. Some of the livers were fixed in 10% formalin, paraffin-embedded, and 4 μm paraffin sections cut and stained with H&E to visualize micrometastases. For some experiments, mice were inoculated with tumor cells stably expressing the GFP gene and the development of hepatic metastases was monitored using live imaging.

Live animal fluorescence optical imaging was performed using a cooled CCD IVIS 13198 camera mounted in a light-tight specimen box (IVIS™; Xenogen). The Living Image® analysis software (Xenogen) was used for acquisition and quantification of signals. The mice were anesthetized, placed onto a warmed stage inside the light-tight box and imaged for 5-10 seconds depending on the time interval following tumor inoculation. The fluorophore excitation and emission filter sets used were λ_{excitation}=445-490 nm and λₑₘᵢₛₛᵢₒₙ=515-575 nm. The fluorescence images shown are real-time unprocessed images and the scale of florescent intensity is shown.

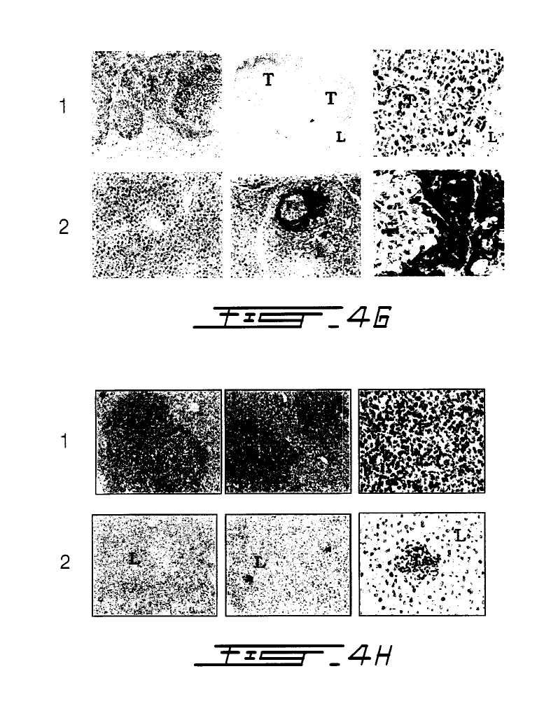

In all mice implanted with BMSC^{sIGFIR933} cells, a marked reduction in the number of hepatic metastases was observed (FIGS. 4A-E, lane 3). The results shown in FIGS. 4A-E demonstrate that in mice injected with H-59 cells 9 days following implantation of BMSC^{sIGFIR933} cells, the median number of hepatic metastases declined by 70, 78, and 80%, respectively, relative to BMSC^{GFP}- and BMSC^{EPO}implanted or untreated control mice (FIGS. 4A and 4B; lanes 2 respectively) and this inhibitory effect was still apparent when tumor cells were inoculated 14 days post BMSC implantation resulting in reductions of 93-95% in the median number of metastases relative to control groups (FIG. 4C, and see representative livers in FIG. 4F). A similar inhibitory effect of BMSC^{sIGFIR933} cells was seen following injection of colon carcinoma MC-38 (FIG. 4D; lane 3) or KM12SM (FIG. 4E, lane 3) cells where the number of metastases declined by 78-82 and by 64%, respectively relative to the indicated control groups. In none of the experiments, was there a significant difference between the number of metastases developed in control BMSC^{GFP} (mock-treated) or untreated mice (FIG. 4A, C-E; lane 2) suggesting that the implantation of BMSC per se, did not have a deleterious (or stimulatory) effect on the development of hepatic metastases. To compare the course of tumor development in mice implanted with BMSC^{sIGFIR933} and control cells, these cells were implemented into athymic nude mice 9 days prior to injection of GFP-tagged H-59 cells and tracked the appearance of a GFP signal in the liver using the Xenogen IVIS® 200 system for noninvasive optical imaging. In all mice implanted with control BMSC, a green fluorescent signal localized to the hepatic region could be detected by day 11 post tumor injection. In mice implanted with BMSC^{sIGFIR933} cells, hepatic tumors were first seen on day 15 post tumor inoculation (1/7 mice) and only 2/7 mice had a detectable GFP signal by day 18, when all the mice were euthanized (FIGS. 4G and I). Post-mortem analysis confirmed that metastases in both groups were confined to the liver and no-extrahepatic metastases were observed (FIG. 4H).

### Example 5

### Reduced Angiogenesis and Increased Tumor-Associated Apoptosis During the Early Stages of Liver Colonization in Mice Producing the Soluble IGF-IR Decoy

The IGF-I receptor is a survival factor and has also been implicated in tumor-induced angiogenesis as a regulator of VEGF production. Angiogenesis is a physiological process involving the growth of new blood vessels from pre-existing vessels. The live imaging results disclosed herein demonstrate that tumor development in BMSC^{sIGFIR933}—implanted mice was significantly delayed and therefore the underlying mechanisms were investigated by comparing tumor-induced angiogenesis in treated and control mice, 6 days post inoculation of GFP+H-59 cells.

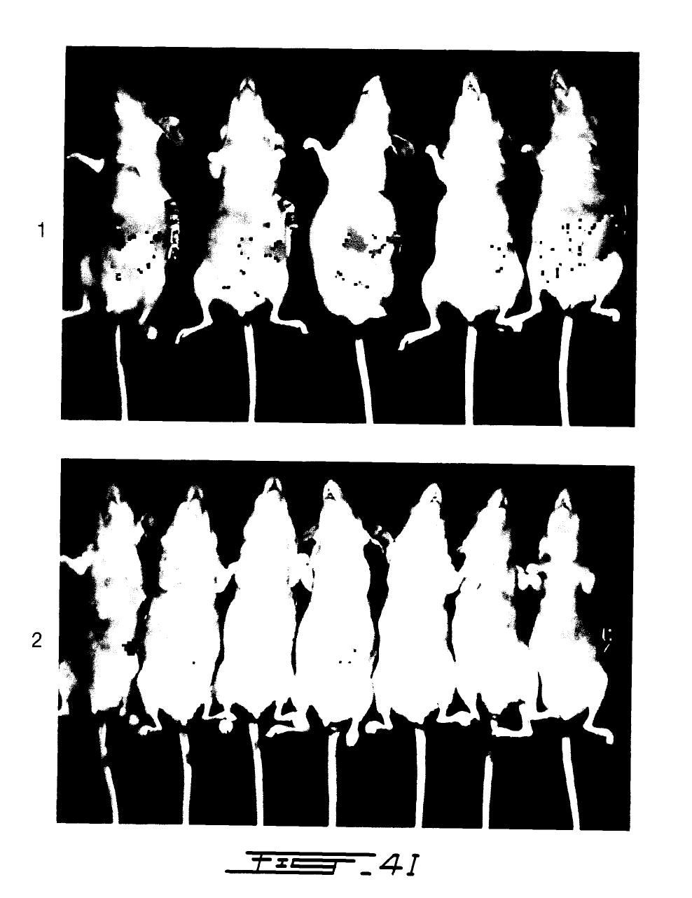

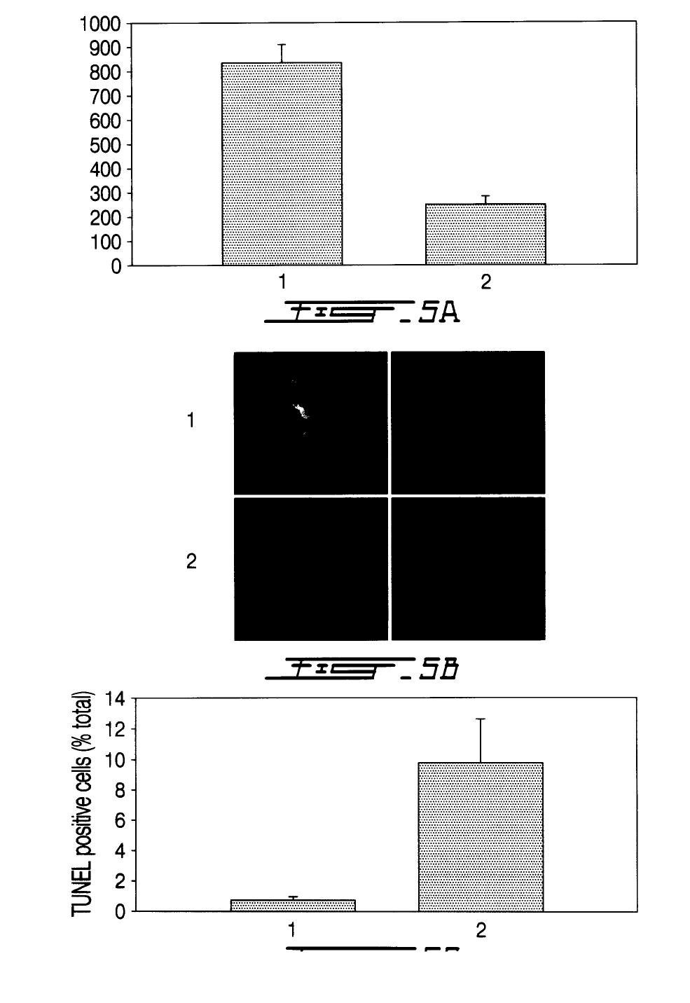

Liver cryostat sections were immunostained with an antibody to the endothelial marker CD31 and an Alexa Fluor⁵⁸⁶ secondary antibody, and the tissue sections were analyzed using confocal microscopy. Quantification of tumor-lesion associated microvessels was carried out using the Zeiss LSM5 image browser software. Results in FIG. 5A show that the number of tumor-associated vessels in mice producing the decoy receptor (second histogram) declined by >3 fold relative to the control group (first histogram). In these mice (second portion of panel), but not in control animals (first portion of panel), numerous micrometastases that were devoid of vascular structures could be observed (FIG. 5B).

To measure the extent of apoptosis within micrometastatic lesions, the livers were removed 6 days following the intrasplenic/portal injection of GFP-tagged H-59 cells, snap frozen and 8 μm cryostat sections prepared. The sections were fixed for 20 min in cold PBS containing 4% paraformaldehyde, washed in cold PBS, and permeabilized with a 0.1% Triton X-100 solution in 0.1% sodium citrate. Apoptotic cells were labeled by a TdT-mediated dUTP nick end-labelling (TUNEL)-based assay, using the in situ cell death detection kit, TMR red (Roche Diagnostics, Laval, Quebec), according to the manufacturer's instructions. Nuclei were stained with 4′,6-Diamidino-2-phenylindole (DAPI). The sections were mounted using the Pro-Long® GOLD anti-fade reagent (Invitrogen Molecular Probes, Burlington, ON Canada). The cells were visualized using the LSM 510 Meta confocal microscope (Carl Zeiss Canada, Toronto, ON) and the images acquired and analyzed with the Zeiss LSM Image Browser program. Images of twenty random fields at a 63× magnification were acquired, the number of red-fluorescent cells and total nuclei per field recorded and the proportion of apoptotic cells calculated as the percentage of red-fluorescent cells per total number of nuclei in each field.

To measure tumor-induced angiogenesis, the mice were inoculated with 10⁵ tumor cells by the inrasplenic/portal route, euthanized 6 days later and the livers perfused via the portal vein with a solution of 4% paraformaldehyde in PBS, excised, fixed in 4% paraformaldehyde for an additional 48 hours and then placed in a solution of 30% sucrose for 4 days prior to preparation of 8 μM cryostat sections. To stain intra-lesional microvessels, the sections were first incubated in a blocking solution containing 0.1% BSA, 5% goat serum and 0.1% Triton×100 in PBS, washed in PBS for 30 min and then incubated, first with a rat anti mouse CD31 antibody (BD Pharmingen, BD Biosciences) for 18 hours at 4° C. and then with an Alexa Fluor⁵⁶⁸ goat anti-rat IgG (Invitrogen™), Burlington), both at a dilution of 1:200. The sections were mounted using the Pro-Long® GOLD anti-fade reagent (Invitrogen) and images acquired and analyzed using confocal microscopy (as above). The number of CD31⁺ vessels/μm² was determined with the aid of the Zeiss LSM Image Browser program using 20 random images of early hepatic micrometases that were acquired at a ×100 magnification.

The TUNEL assay revealed that the number of apoptotic tumor cells within these hepatic lesions increased by 16 fold in sIGF-IR producing cells as compared to control animals (FIGS. 5C-D). Taken together, these findings demonstrate that tumor growth in these mice was abrogated due to reduced vascularization and enhanced tumor cell death during the early stages of hepatic colonization.

### Example 6

### Reduced Liver Metastasis in sIGFIR Injected Mice

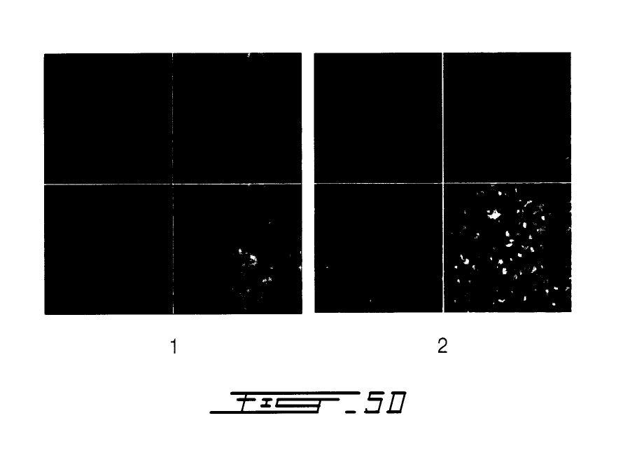

Soluble receptor (sIGFIR) cDNA was expressed in the packaging HEK293 cell line that was genetically engineered for large-scale production of lentiviral vector-derived proteins, based on the use of the cumate switch system, as described in Broussau et al., Molecular Therapy 16(3):500-507 (2008)). Forty 293SF-rcTA-Cym clones were screened by spot blotting and 2 clones that produced high sIGFIR levels were selected for expansion. Selected clones were expanded in suspension cultures in the presence of Cumate (the inducer) for 4-5 days. Supernatants of these cells were then concentrated 20-25 fold using the Minimate™ Tangential Flow Filtration (TFF) system and the sIGF1-R protein was purified using FPLC and an Ni-NTA column. Purity was verified by PAGE and Western blotting (FIGS. 6A, 6B).

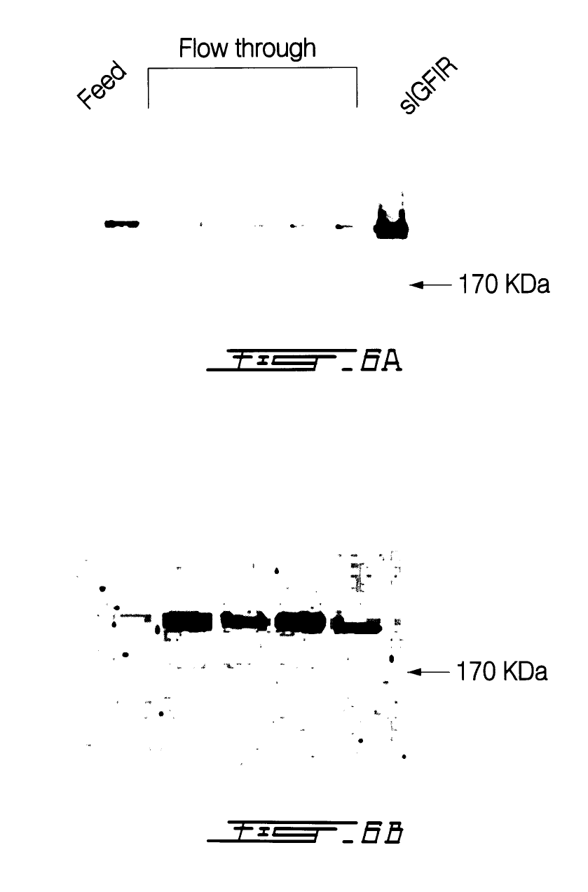

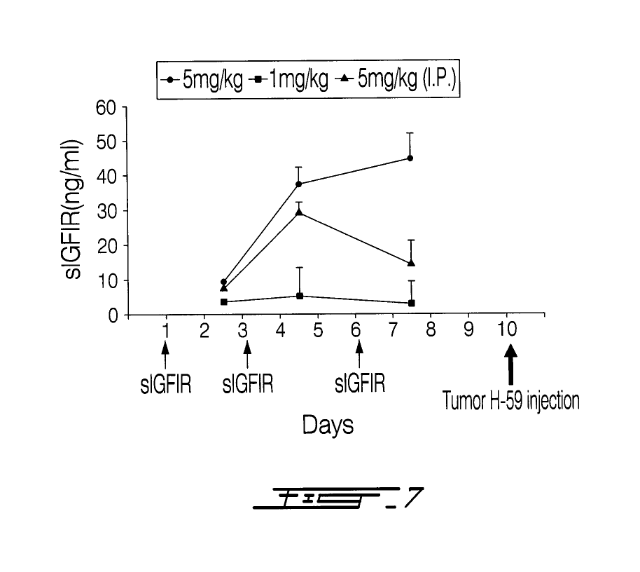

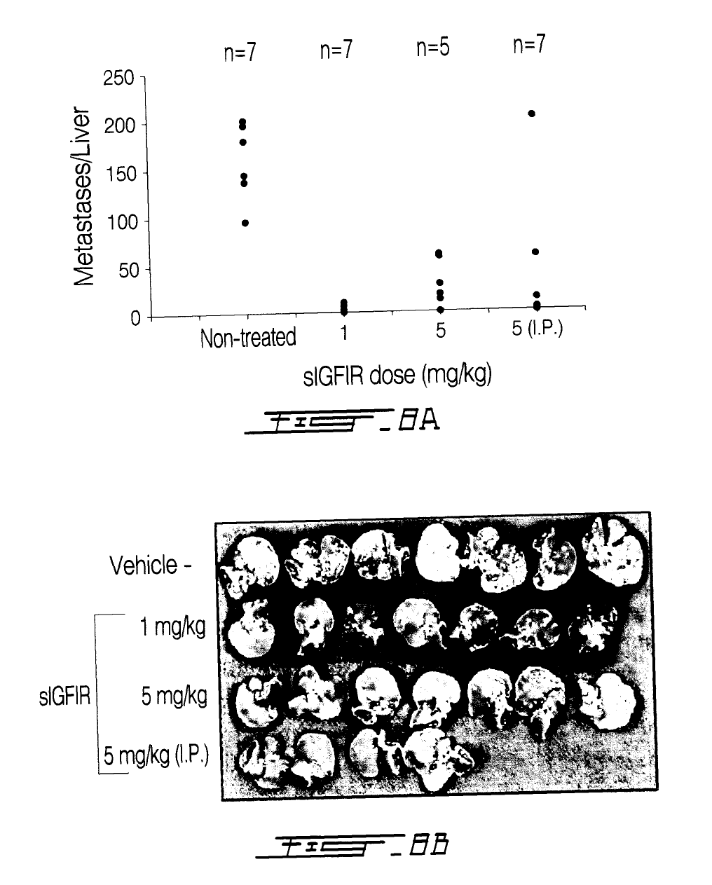

Purified sIGFIR was injected into mice (i.v. or i.p.) on days 1, 3 and 6, at 1 mg/kg, 5 mg/kg, or 5 mg/kg i.p. Plasma levels of sIGFIR after injection are shown in FIG. 7. Intrasplenic/portal inoculation of tumor H-59 cells was carried out on day 10. The effect on liver metastases of sIGFIR was evaluated 14 days following the injection of tumor cells. As shown in FIGS. 8A and 8B, liver metastasis was reduced in sIGFIR injected mice.

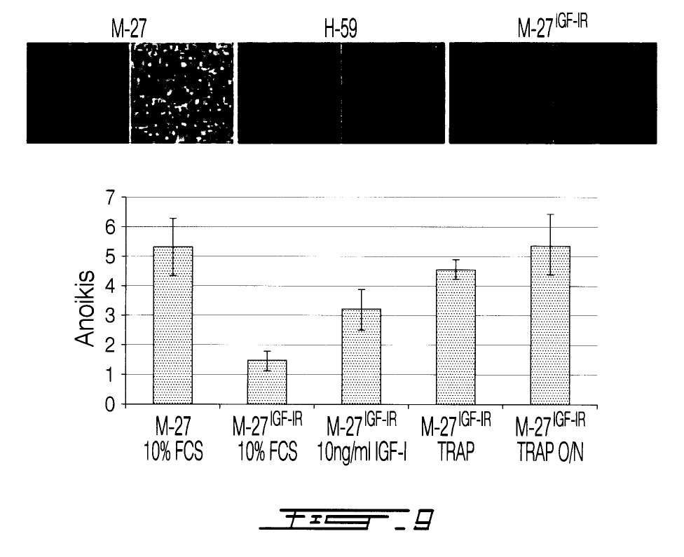

Tumor cells were also incubated in vitro with sIGFIR. As shown in FIG. 9, the incubation of tumor cells in vitro with the soluble IGF-IR increased anoikis, a form of apoptosis induced by cell detachment, of the tumor cells.

These experiments represent the first demonstration that administration of the purified soluble sIGFIR reduced metastasis and induced apoptosis. Previous studies used a cell-based therapy (see for example Samani et al. Cancer Research 64: 3380-3385, 2004) in place of administration of the soluble peptide as demonstrated here.

All references and documents referred to herein are hereby incorporated by reference in their entirety.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of inhibiting angiogenesis in a subject having an angiogenic associated disorder comprising administering to said subject a therapeutically effective amount of a soluble IGF-IR protein comprising the extracellular domain of IGF-IR having the amino acid sequence of SEQ ID NO: 3 or a biologically active fragment thereof.

2. The method of claim 1, wherein said soluble IGF-IR protein forms the tetrameric structure of SEQ ID NO: 3.

3. The method of claim 1, wherein said soluble IGF-IR protein comprises SEQ ID NO: 1 or a biologically active fragment or analog thereof.

4. The method of claim 1, wherein said soluble IGF-IR protein consists of SEQ ID NO: 1.

5. The method of any one of claims 1-4, wherein said angiogenic associated disorder is cancer.

6. The method of claim 1, wherein said angiogenic associated disorder is tumor metastasis, colorectal carcinoma, lung carcinoma, or hepatic cancer.

7. The method of claim 6, wherein said hepatic cancer is liver metastasis.

8. The method of claim 1, further comprising administering the soluble IGF-IR protein in combination with another angiogenesis inhibitor.

9. A method of inhibiting angiogenesis in a subject having an angiogenic associated disorder comprising administering to said subject a stromal cell genetically modified to express a soluble IGF-IR protein comprising the extracellular domain of IGF-IR having the amino acid sequence of SEQ ID NO: 3 or a biologically active fragment thereof.

10. The method of claim 9, wherein said soluble IGF-IR protein forms the tetrameric structure of SEQ ID NO: 3.

11. The method of claim 9, wherein said stromal cell is a bone marrow derived mesenchymal stromal cell.

12. The method of claim 9, wherein said soluble IGF-IR protein comprises SEQ ID NO: 1 or a biologically active fragment or analog thereof.

13. The method of claim 9, wherein said soluble IGF-IR protein consists of SEQ ID NO: 1.

14. The method of any one of claims 9-13, wherein said angiogenic associated disorder is cancer.

15. The method of claim 9, wherein said angiogenic associated disorder is tumor metastasis, colorectal carcinoma, lung carcinoma or hepatic cancer.

16. The method of claim 15, wherein said hepatic cancer is liver metastasis.

17. The method of claim 9, further comprising administering the soluble IGF-IR protein in combination with another angiogenesis inhibitor.

18. Use of a soluble IGF-IR protein comprising the extracellular domain of IGF-IR having the amino acid sequence of SEQ ID NO: 3 or a biologically active fragment thereof for inhibiting angiogenesis in a subject having an angiogenic associated disorder.

19. The use of claim 17, wherein said soluble IGF-IR protein forms the tetrameric structure of SEQ ID NO: 3.

20. The use of claim 18 or 19, wherein said soluble IGF-IR protein comprises SEQ ID NO: 1 or a biologically active fragment or analog thereof.

21. The use of any one of claims 18-20, wherein said soluble IGF-IR protein consists of SEQ ID NO: 1.

22. The use of claim 21, wherein said angiogenic associated disorder is cancer.

23. The use of any one of claims 18-22, wherein said angiogenic associated disorder is tumor metastasis, colorectal carcinoma, lung carcinoma or hepatic cancer.

24. The use of claim 23, wherein said hepatic cancer is liver metastasis.

25. The use of any one of claims 18-24, further comprising administering the soluble IGF-IR protein in combination with another angiogenesis inhibitor.

26. Use of a stromal cell genetically modified to express a soluble IGF-IR protein comprising the extracellular domain of IGF-IR having the amino acid sequence of SEQ ID NO: 3 or a biologically active fragment thereof for inhibiting angiogenesis in a subject having an angiogenic associated disorder.

27. The use of claim 26, wherein said soluble IGF-IR protein forms the tetrameric structure of SEQ ID NO: 3.

28. The use of claim 26 or 27, wherein said stromal cell is a bone marrow derived mesenchymal stromal cell.

29. The use of any one of claims 26-28, wherein said soluble IGF-IR protein comprises SEQ ID NO: 1 or a biologically active fragment or analog thereof.

30. The use of any one of claims 26-29, wherein said soluble IGF-IR protein consists of SEQ ID NO: 1.

31. The use of any one of claims 26-30, wherein said angiogenic associated disorder is cancer.

32. The use of claim 31, wherein said angiogenic associated disorder is tumor metastasis, colorectal carcinoma, lung carcinoma or hepatic cancer.

33. The use of claim 32, wherein said hepatic cancer is liver metastasis.

34. The use of any one of claims 26-33, further comprising administering the soluble IGF-IR protein in combination with another angiogenesis inhibitor.

35. The method of claim 8, wherein the soluble IGF-IR protein and the other angiogenesis inhibitor are administered concomitantly or sequentially.

36. The method of claim 1 wherein an angiogenic associated disorder is prevented or treated in the subject.

37. The method of claim 1 wherein tumor metastasis, colorectal carcinoma, lung carcinoma or hepatic cancer is prevented or treated in the subject.

38. A pharmaceutical composition for inhibiting angiogenesis in a subject, comprising a soluble IGF-IR protein comprising the extracellular domain of IGF-IR having the amino acid sequence of SEQ ID NO: 3 or a biologically active fragment thereof; and a pharmaceutically acceptable carrier.

39. Use of a soluble IGF-IR protein comprising the extracellular domain of IGF-IR having the amino acid sequence of SEQ ID NO: 3 or a biologically active fragment thereof in the manufacture of a medicament for inhibiting angiogenesis in a subject having an angiogenic associated disorder.

40. The method of claim 36 or 37, or the pharmaceutical composition of claim 38, or the use of claim 39, wherein said soluble IGF-IR protein forms the tetrameric structure of SEQ ID NO: 3.

41. The method of claim 36 or 37, or the pharmaceutical composition of claim 38, or the use of claim 39, wherein said soluble IGF-IR protein comprises SEQ ID NO: 1 or a biologically active fragment or analog thereof.

42. The method of claim 36 or 37, or the pharmaceutical composition of claim 38, or the use of claim 39, wherein said soluble IGF-IR protein consists of SEQ ID NO: 1.

43. The method of claim 36, or the use of claim 39, wherein said angiogenic associated disorder is cancer.

44. The method of claim 36, or the use of claim 39, wherein said angiogenic associated disorder is tumor metastasis, colorectal carcinoma, lung carcinoma or hepatic cancer.

45. The method of claim 36, or the use of claim 39, wherein said angiogenic associated disorder is liver metastasis.

46. The method or use of any one of the preceding claims wherein said soluble IGF-IR protein retains the disulfide bonds of SEQ ID NO:3 and/or high affinity ligand binding.

47. The method of claim 1 wherein the soluble IGF-IR protein is administered via injection.

48. The method of claim 47, wherein the injection is intravenous or intraperitoneal.

49. The method of claim 35, wherein the soluble IGF-IR protein is administered via injection.

50. The method of claim 49, wherein the injection is intravenous or intraperitoneal.


SEQUENCE LISTING (4 sequences)

SEQ ID NO: 1 - protein, 933 aa, Artificial Sequence
  sIGFIR933 protein sequence
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENF

SEQ ID NO: 2 - DNA, 2799 bp, Artificial Sequence
  cDNA fragment
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttc

SEQ ID NO: 3 - DNA, 4104 bp, Homo Sapiens
  Full length IGF-IR
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctga

SEQ ID NO: 4 - protein, 1367 aa, Homo Sapiens
  Full length IGF-IR
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC